(19) United States
(12) Patent Application Publication
(10) Pub. No.: US 2022/0061776 A1
(43) Pub. Date: Mar. 3, 2022

(54) SYSTEM AND METHOD OF REGISTERING AND COMMUNICATING USER CONDITION TO A REMOTE DEVICE

(71) Applicant: Slim Souissi, San Diego, CA (US)

(72) Inventor: Slim Souissi, San Diego, CA (US)

(73) Assignee: Ospitek, Inc., San Diego, CA (US)

(21) Appl. No.: 17/002,066

(22) Filed: Aug. 25, 2020

(51) Int. Cl. A61B 5/00

(52) U.S. Cl. CPC A61B 5/747; A61B 5/7475; A61B 5/4824; A61B 5/746; A61B 5/7455; A61B 5/002; A61B 5/7405; A61B 5/742; A61B 5/0004

(57) ABSTRACT

A system for registering and communicating user condition to a remote device is provided. The system comprises a portable device comprising a processor and a memory, a transmitter, a user interface that displays a plurality of selectable objects, each object associated with a level of user condition, and an application stored in the memory. When executed on the processor, the application receives a message associated with a user entry to a first selectable object of the plurality of selectable objects. The application also determines a first level of the user condition based on analysis of the first message. The application also sends, via the transmitter, electronic material describing the first level to a server requesting action associated with the first level.

### FIELD OF THE APPLICATION

The present disclosure is in the field of healthcare devices. More specifically, the present disclosure teaches systems and methods of a patient using a handheld device to communicate levels of pain to a remote server and to request appropriate action.

### BACKGROUND

Pain is a common reason that patients contact physicians, hospitals and other healthcare providers. Some medical professionals refer to pain as the ‘fifth vital sign.’ Pressing needs exist to improve pain management outcomes.

A further need is to maximize pain management scores on surveys such as the Hospital Consumer Assessment of Healthcare Providers and Systems Survey. This survey, referred to as the HCAHPS survey, is a standardized survey instrument and data collection methodology that has been in use since 2006 to measure patients' perspectives of hospital care.

Measurement of brain activity is a means for continuously and automatically measuring patient pain. Significant research has been conducted in this area to date. Much of this research deals with early stage unproven solutions that are expensive and presently difficult to implement. Health care providers lack simple and cost-effective systems that provide for reasonably accurate pain measurement. No system exists that does not require staff intervention. No system exists that relies on patients' personal feedback about pain.

Patients in healthcare facilities historically use a call button device to alert a nurse or other staff member remotely when they need help. Such a device provided in the part art may consist of a single button attached to a cable located near the patient's bed or in the bathroom. The single-button device allows patients to instantaneously and remotely communicate their need for help. When the button is pressed, a signal alerts staff at the nurse's station, and usually, a nurse or nurse assistant responds to the call.

Some systems also are equipped with speakers and microphones and allow the patient to speak directly to the nurse. Other such devices simply beep or buzz at the nurse station, requiring a staffer to physically visit the patient's room to determine the patient's needs. This previously provided device acts like a simple communication mechanism between patient and nurse without providing details about the patient's needs.

Other previous implementations may provide more sophisticated systems and methods for measuring patient pain. A firm named PainQx, Inc. has developed an advanced approach to objectively measure pain. A platform provided by this approach relies on neural activity detected from a patient's brain. Processing and decoding of this activity is conducted using proprietary algorithms. The output of the PainQx implementation is a scaled pain and neurological side effects biomarker that is directly correlated to a patient's pain state. But this implementation is still in its trial stages.

The lack to date of inexpensive, reliable and efficient pain measurement systems has a continuing negative impact on overall patient satisfaction. Hence a need exists for simple and cost-effective systems and methods for automatically registering and monitoring patients' pain during their stays at healthcare facilities.

### BRIEF DESCRIPTION OF THE DRAWINGS

FIG. 1 is a diagram of a system for registering and communicating user condition to a remote device in accordance with an embodiment of the present disclosure.

FIG. 2 is a diagram of a system for registering and communicating user condition to a remote device in accordance with an embodiment of the present disclosure.

FIG. 3 is a diagram of a system for registering and communicating user condition to a remote device in accordance with an embodiment of the present disclosure.

FIG. 4 is a diagram of a system for registering and communicating user condition to a remote device in accordance with an embodiment of the present disclosure.

### DETAILED DESCRIPTION

Systems and methods described herein provide a hand-held pain registration calling device for hospital patients to use in communicating levels of pain to a nursing stations and other areas. The device has a plurality of selectable keys or buttons, each representing a different level of pain. For example, the device may have six such buttons. A first button may represent mild pain. The second through fifth buttons represent increasing levels of pain. The sixth button may represent severe or excruciating pain.

The patient in his/her hospital room or bathroom manually grasps the device and presses the button appropriate for his/her level of pain. The device sends the patient's selection to a server. The server posts the selected pain level, the patient's room number, and the date and time to a television screen or monitor and to client devices viewable by nurses and others.

The server also sends audible, visual, and/or vibratory alerts to some client devices carried by nurses and others. The client devices may be used to visualize patient pain profiles over time and generate analytics regarding pain levels as a function of type of surgery, surgeon, nurse, time of day, patient age, gender and other parameters.

The server also updates the calling patient's chart and other records with pain level information. The stored pain information is used for further analysis and correlation to patient satisfaction surveys.

Systems and methods provided herein may further increase frequency of registration of patient pain level. The need for unnecessary and cumbersome paper work currently used by hospitals may also be reduced with the consequent increase in nurse productivity.

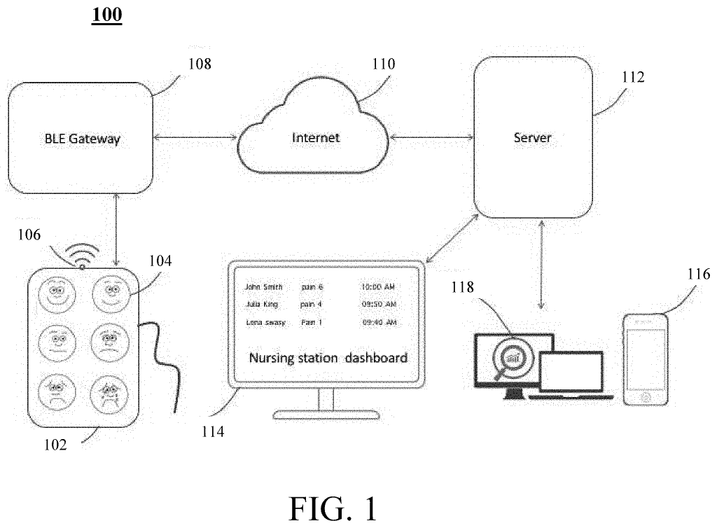

Turning to the figures, FIG. 1 is a diagram of a system for registering and communicating user condition to a remote device in accordance with an embodiment of the present disclosure. FIG. 1 illustrates components of a system 100 comprising a calling device 102, a button 104, a transmitter 106, a BLE Gateway 108, and the Internet 110. System 100 also comprises a server 112, a nursing station dashboard 114, and client devices 116, 118. While not illustrated, software applications central to systems and methods provided herein execute on both the calling device 102 and the server 112.

A patient in his/her room manually holds the calling device 102 and presses the button 104 corresponding to his/her level of pain. The transmitter 106 sends information including at least the selected level of pain, the patient's room number, and the date and time of the transmission to the server 112 via the BLE Gateway 108 and the Internet 110.

The server 112 posts the information to the dashboard 114 located at a nearby nursing station and sends vibratory, audible, and visual alerts to client devices 116, 118 used by nurses and others. The server 112 also updates the patient's chart and other records.

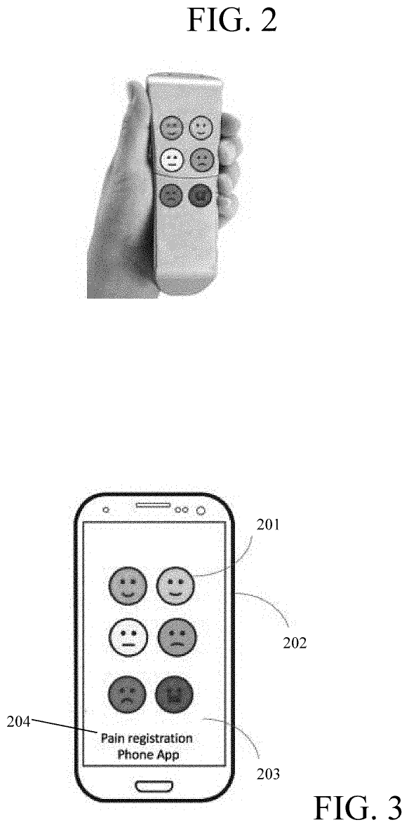

In an embodiment, the calling device 102 has quantity six of buttons 104, each button 104 selectable for a different level of pain. Each button 104 may have an image of a human face, each image exhibiting a facial expression commensurate with the pain level to be reported via pressing of the particular button 104. FIG. 2 is a view of a typical calling device 102 of the present disclosure.

FIG. 3 is a close-up view of the calling device 102 of the system 100, depicted as calling device 202 in FIG. 3. Button 201 is depicted as is the general interface 203. A pain registration phone app 204 is stored in and executes on the calling device 202. The app 204 processes input and provides such processed inputs to the transmitter 106 of the system 100.

In an embodiment, the calling device 102 may be replaced by a smartphone and a smartphone application. The smartphone may communicate with the server 112 via Bluetooth Low Energy functionality or a 4G/5G cellular network. In embodiments, the calling device 102 may feature hardware and functionality provided by devices commercially known as Siri and Alexa.

In an embodiment, the calling device 102 or a smartphone may be used to convey more detailed information about patient pain. The calling device 102 may provide an image of a human body with pre-programmed points representing areas of potential pain suffered by the patient. The patient may press at least one of the points to initiate a message being sent to the server 112 as described above. The amount of time the patient (or medical staff) holds the button represents the level of pain experienced by the patient. Information about the particular point selected on the depicted body as well as the amount of time the point is pressed is sent to the server 112 along with the patient's room number and the date and time.

The image of the human body depicted may include both front views and back views provided simultaneously in a single view or separately and available by toggling back and forth. The views include numerous pre-programmed points such as knees, elbows, stomach, buttocks, and heart, for example, areas of concentration of pain about which the user wishes to report. The user can report more than one area of pain during a session. Nurses and others viewing the dashboard 114 at their stations may be able to diagnose quickly the patient's condition based on analysis of the patient's reported combinations of pain areas and reported intensity of pain in each area

A patient in an example may hold a selected area for 4 seconds. An icon shown as a smiley face, for example, may be displayed. The icon may represent user pain level 4 which is sent to the server 112 via the transmitter 106. Alternatively, a pull-down menu may be used to designate a pain level for the selected pain area. Color coded dots may also be used by the patient to select the pain level.

Based on the selected pain area and the severity of the pain, a notification message can be sent to the appropriate health care specialist as well as posted to the dashboard 114 as described above. For instance, an expected headache after surgery would be reported to the resident nurse and may be treated by conventional pain killers. However, a severe chest pain after heart surgery may trigger an immediate notification to the cardiologist in charge of the patient. The interface may also provide means to cancel the registration of pain in case the patient has made a mistake.

In an embodiment, a stress ball may replace the calling device 102. The stress ball is equipped with a BLE radio to communicate the amount of pressure manually exerted by the patient on the ball. Pressure levels on the stress ball are calibrated to represent pain levels. Based on the amount of pressure applied by the patient, a smiley face indicator or other graphic is shown to the patient. If the patient presses hard enough for smiley face yellow, which may be representative of pain level 4, to be exhibited, the level 4 will be communicated to the server 112 upon the patient releasing the ball or as soon as the pressure level is observed to be unchanged over a predefined period of time. The smiley face indicator could be a simple colored LED or a small color display integrated within the ball.

The indicator may be an image other than a smiley face. The indicator may be any graphic whose appearance changes given different levels of manual pressure exerted on the stress ball.

Instead of pain, the connected stress ball can be used to communicate the level of stress experienced by the patient. In this case, levels of stress should be created and made visible to the patient so as he/she presses the ball the appropriate level appears. Upon the release of the stress ball, the stress level is communicated to the server 112.

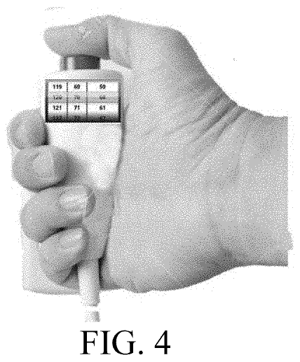

In an embodiment, the calling device 102 may not have selectable buttons as shown in FIG. 1. Instead the calling device 102 may feature a wheel that the patient may turn to select his/her pain level. Once the pain level is selected in this manner, the patient may push a single button to send a message with this information as well as the patient's room number and the date/time to the server as described. The server 112 then proceeds as previously described. Such a calling device 102 with a selectable wheel is depicted in FIG. 4.

The connection of the calling device 102 to the server 112 may be wireless, hard wired or a combination thereof. The wireless connection may be bluetooth, z-wave, zigbee, Lora, Sigfox of narrow band IoT, or cellular 2G, 3G, 4G, 5G. The client device 116, 118 may be a special nursing terminal, a tablet, a desktop computer, a mobile phone, or a smartphone. The nursing station dashboard 114 may be a television screen or other monitor. The server 112 performs analytics on received pain signals from multiple patients. The pain level is further categorized based on its type.

In an embodiment, the nurse or other provider of care that receives notifications from the server 112 may depend on the type of pain selected and on the level of pain selected. Based on categorizations of pain levels by type of pain and intensity of pain, different nurses and other personnel will receive different types of alerts. Such variations of alerts may depend on other factors including staffing levels at the hospital, availability of equipment and other facilities, and overall workload at the hospital. The server 112 may communicate with central systems of the hospital to send and receive data regarding patient conditions received from the calling devices 102, availability of hospital resources to handle such conditions, and availability of facilities and equipment.

1) A system for registering and communicating user condition to a remote device, comprising:
a portable device, comprising:
a processor and a memory;
a transmitter;
a user interface that displays a plurality of selectable objects, each object associated with a level of user condition; and
an application stored in the memory that when executed on the processor:
receives a message associated with a user entry to a first selectable object of the plurality of selectable objects,
determines a first level of the user condition based on analysis of the first message, and
sends, via the transmitter, electronic material describing the first level to a server requesting action associated with the first level.

2) The system of claim 1, wherein the user entry is made by a patient situated at a healthcare facility.

3) The system of claim 1, wherein the user condition is pain.

4) The system of claim 1, wherein each level of user condition associated with the selectable objects describes a differing level of user pain.

5) The system of claim 1, wherein upon receipt of the message, the application additionally sends an alert to a manned station.

6) The system of claim 1, wherein the requested action associated with the first level is caregiver response to the user.

7) The system of claim 1, wherein the user interface one of additionally and alternatively comprises at least one image of a human body with selectable areas for reporting a user condition associated with an area within the at least one image.

8) The system of claim 7, wherein an amount of time user pressure is exerted against a selected area within the image indicates level of user condition.

9) The system of claim 8, wherein information describing the selected area and an amount of time of user pressure is sent, via the transmitter, to the server.

10) A system for registering and communicating user condition to a remote device, comprising:
a server, comprising:
a processor and a memory; and
an application stored in the memory that when executed on the processor:
receives a message containing material describing a first level of a user condition, the user condition associated with a user of a portable device,
based on the first level, posts a first notification to at least one display viewable by personnel responsible for action associated with the first level, and
transmits at least one of an auditory, vibratory, and visual alert to at least one device accessible by the personnel.

11) The system of claim 10, wherein the application further updates a record of the user with at least the material.

12) The system of claim 10, wherein the user is a patient situated in a healthcare facility.

13) The system of claim 10, wherein the material describes at least a first level of a user condition.

14) The system of claim 10, wherein the user condition is pain.

15) A method for registering and communicating user condition to a remote device, comprising:
a stress ball receiving a first exertion of manually applied pressure;
the ball activating a pressure measurement device to determine a measurement of the applied pressure;
the ball, based on the determined measurement, activating local radio functionality; and
the ball transmitting, via the radio functionality, the measurement to a server requesting action.

16) The method of claim 15, wherein the first exertion is received from a user of the ball to indicate a level of a user condition.

17) The method of claim 16, wherein the user condition is pain and the user is a patient in a healthcare facility.

18) The method of claim 15, wherein a greater exertion of manually applied pressure is associated with a higher level of pain than levels of pain associated with lesser exertions of pressure by the user.

19) The method of claim 15, wherein the radio functionality is Bluetooth Low Energy functionality.

20) The method of claim 15, wherein the requested action is action by personnel responsible for care of the user.
